(19) United States
(12) Patent Application Publication
(10) Pub. No.: US 2022/0346837 A1
(43) Pub. Date: Nov. 3, 2022

(54) NON-INVASIVE EGG RETRIEVAL PROTOCOLS AND METHODS

(71) Applicant: Butterfly Biosciences, Inc., Berkeley, CA (US)

(72) Inventors: Sam Najmabadi, Beverly Hills, CA (US); Moses Cesario, Oakland, CA (US); Alexander Nadal, Los Angeles, CA (US); Steven T. Nakajima, Broomfield, CO (US); José Luis Rivas, Punta de Mita (MX); Ricardo Peraza, Bucerias (MX)

(21) Appl. No.: 17/734,799

(22) Filed: May 2, 2022

Related U.S. Application Data
(60) Provisional application No. 63/258,422, filed on May 3, 2021.

(51) Int. Cl. A61B 17/435; A61P 15/08; A61K 38/09; A61K 38/24; A61K 9/00; C12N 5/073

(52) U.S. Cl. CPC A61B 17/435; A61P 15/08; A61K 38/09; A61K 38/24; A61K 9/0034; C12N 5/0604; A61B 2017/4216

(57) ABSTRACT

Methods for non-invasive retrieval of viable (i.e., able to be fertilized) oocytes from the uterus via a combination of unique ovarian stimulation and uterine lavage technology. These methods may eliminate the need for an invasive surgical procedure. Embodiments include the use of a novel hormonal protocol, including the use of injectable gonadotropins, to induce the release of immature oocytes from the ovaries of a patient. The immature oocytes mature as they transit the fallopian tubes such that mature, viable oocytes may be collected from the uterus via a uterine lavage procedure. Further embodiments include the use of a novel hormonal protocol, including the administration of Prostaglandins, Estradiol, and/or Progesterone Blockers, to accelerate transport of oocytes through the fallopian tubes. Further embodiments include kits for uterine lavage, compositions suitable for triggering and inducing superovulation, and compositions suitable for accelerating tubal transport of mature oocytes in a female patient.

In the United States, there are approximately 1,400 reproductive endocrinologists (REs) and 450 in vitro fertilization (IVF) clinics to serve the rapidly growing segment of patients desiring fertility treatments. This small footprint creates insufficient throughput of fertility services, accordingly assisted reproduction cycles remain stagnant at approximately 250K assisted reproduction cycles per year despite millions of people needing treatment. It is estimated that <5% of the United States' fertility needs are being met today and without new methodologies to make assisted reproduction easier and automated, the current infrastructure will continue to fail the millions of people seeking treatment.

One of the many bottlenecks is the need for an oocyte retrieval. The oocyte retrieval is a critical component in the IVF, or egg freezing, process where mature, unfertilized oocytes are aspirated directly from ovarian follicles. This is an invasive surgical procedure requiring anesthesia (or conscious sedation), an operating room, and a highly specialized staff (physicians, nurses, embryology personnel) to perform the oocyte retrieval. During the procedure, a thin needle guided by an ultrasound probe is directed into the ovary where the eggs are aspirated, and then transferred to the embryology lab for identification. Mature oocytes are cryopreserved for future use, or fertilized with sperm. The RE performing the aspiration uses real-time ultrasound imaging to aspirate the follicles and avoid puncture of the intestines or major blood vessels. Due to the complexity, only REs are qualified to perform this procedure and spend three additional years in a RE fellowship after a four year residency in obstetrics and gynecology. If a simplified method of performing the aspiration was available, more doctors could perform the procedure and assisted reproduction could be made available to millions more.

Various embodiments include methods for non-invasive retrieval of viable (i.e., able to be fertilized) oocytes from the uterus via a combination of unique ovarian stimulation and uterine lavage technology. These steps eliminate the need for an invasive surgical procedure. Uterine lavage refers to a rinsing of the uterine cavity to collect samples (e.g., embryos, oocytes, sperm and cells from the ovaries, fallopian tubes, endometrial cavity and cervix) from the reproductive system.

Challenge of Uterine Lavage

The primary challenge of using uterine lavage for egg retrieval is the viability of the oocytes at the time of recovery; if eggs are not viable they have no clinical utility. In natural cycles (without ovarian stimulation), mature oocytes are ovulated from the ovaries and remain viable for 24 hours after ovulation, but it takes approximately four days (or 96 hours) for the eggs to reach the uterus from the fallopian tubes. At the time that oocytes reach the uterus, they are no longer viable. This is corroborated by Munne et al (2020) where unfertilized oocytes were collected from the uterus through uterine lavage but could not be fertilized in-vitro.

In the historical work of Munne et al, injectable gonadotropins were administered to stimulate multiple follicles in the ovaries for 9-12 days. Use of a gonadotropin antagonist (GnRH antagonist) was administered to keep oocytes in the ovaries and prevent early ovulation. When the oocytes were of sufficient maturity, an ovulatory trigger was administered to mature the eggs and fixed the time for intrauterine insemination (IUI), and the subsequent uterine lavage procedure. Five days after IUI, a uterine lavage procedure was scheduled when appropriately developed embryos (now called blastocysts) were expected to be in the uterus.

Novel Hormonal Stimulation Protocol—Early Follicular Release

The inventors have developed a novel hormonal protocol to make possible the collection of viable oocytes from the uterus after a uterine lavage cycle. This protocol involves use of injectable gonadotropins over a 3-9 day period, and may also include a GnRH antagonist to prevent early unsynchronized release of oocytes. Immature oocytes (i.e. germinal vesicle and metaphase I) are released from the ovaries and traverse the fallopian tubes en route to the uterus. During transit, immature oocytes will mature in the fallopian tubes and reach the uterus at such time when the oocytes are mature and capable of fertilization. The uterine lavage should occur approximately 3-7 days after the ovulatory trigger.

The optimized protocol for non-invasive oocyte retrieval is as follows:
1. (Optional step) Start oral contraceptives (OCPs) on cycle day (CD) 1-3 and suppress ovaries for a minimum of 10-21 days.
2. Stop OCPs and on the 5^{th} day after stopping the OCPs, or once menses starts, begin superovulation to lead to the early production and release of many (for example, three or more) oocytes in one menstrual cycle. Superovulation is induced using injectable gonadotropins (FSH and LH) to stimulate maturation of multiple oocytes (e.g., Gonal F or Follistim or Menopur) at a range of 37.5-300 IU over 3 to 11 days, for example, at 150 IU/day over 3 days followed by 100 IU/day over 6 days.
3. Trigger with human chorionic gonadotropin (hCG), at a range of 1,000-10,000 IU, for example, 5,000 IU, and/or Lupron (4 mg) when dominant follicle (DF) is at or between 10-18 mm in diameter. This can be confirmed by ultrasound to image the ovaries. The released oocytes are captured in the open end of the Fallopian tube and move towards the uterine cavity naturally after ovulation. The transit time to the uterus is approximately 3-7 days from the trigger, such that when the oocytes reach the uterus they will be mature. They will mature in transit through the fallopian tubes.
4. Perform uterine lavage approximately 3-7 days after trigger. During the lavage Prostaglandins may be administered into the uterus to close the fallopian tube ostium.
5. If oocytes are immature, will culture for in vitro maturation (IVM)
6. If a metaphase II (MII) oocyte is identified after uterine lavage, plan intracytoplasmic sperm injection (ICSI) with partner or donor sperm
7. Confirm fertilization by the identification of a zygote (single cell embryo). Embryos are further cultured to the blastocyst stage (5-7 days). Blastocysts are then biopsied for preimplantation genetic testing for aneuploidy and subsequent cryopreservation. Alternatively, non-invasive genetic and non-genetic tests can be performed using the culture media collected from IVF such as non-invasive preimplantation genetic testing for a aneuploidy, (NiPGT-A), Metabolomics, or Proteomics.
8. Embryo transfer of a previously cryopreserved euploid blastocyst for the establishment of an intrauterine pregnancy (at a later date).

Novel Hormonal Stimulation Protocol—Accelerated Tubal Transport

The inventors have also developed an additional novel hormonal protocol to make possible the collection of viable oocytes from the uterus after a uterine lavage cycle. This protocol involves use of injectable gonadotropins over a 3-11 day period, applying a GnRH antagonist to prevent early ovulation, and application of an ovulatory trigger at a follicular diameter size of >10 mm. Approximately 36 hours after trigger, mature and immature oocytes (i.e. germinal vesicle and metaphase I) are released from the ovaries and traverse the fallopian tubes en route to the uterus. Approximately 0-36 hours after ovulation trigger, either Prostaglandins, Estradiol, and/or Progesterone Blockers will be administered to accelerate fallopian tube transport. Prostaglandins, Estradiol and/or Progesterone Blockers will increase ciliary movement and smooth muscle contraction to promote movement of the oocytes through the fallopian tubes. It is important to keep estrogen elevated after ovulation because it promotes ciliogenesis, increased tubal fluid, ciliary movement, and smooth muscle contraction. Progesterone levels, however, should be minimized after ovulation as progesterone inhibits ciliary movement and relaxes smooth muscle tissue. This is counterintuitive as in natural cycles and in IVF high estrogen is important for events prior to ovulation such as follicular growth and ovulation; after ovulation estrogen decreases and progesterone increases. Progesterone is involved in smooth muscle relaxation, ciliogenesis inhibition and prepares the endometrium for implantation and will have the opposite effects to estrogen in terms of fallopian tube transport. The inventors have a unique need to reverse the traditional endocrine profile of estrogen/progesterone after ovulation to make possible non-surgical oocyte retrieval. This protocol also has the additional benefit of creating a thinner endometrial layer which is a simpler environment from which to lavage from.

With the above modification, fallopian tube transport will be complete over 10-100 hours after ovulation trigger and oocytes reach the uterus at such time when the oocytes are mature and capable of fertilization. The uterine lavage could occur approximately 24-108 hours after the ovulatory trigger.

The optimized protocol for non-invasive oocyte retrieval is as follows:
9. (Optional step) Start oral contraceptives (OCPs) on cycle day (CD) 1-3 and suppress ovaries for a minimum of 10-21 days
10. Stop OCPs and on the 5^{th} day after stopping the OCPs, begin superovulation to lead to the early production and release of many (for example, three or more) oocytes in one menstrual cycle. Superovulation is induced using injectable gonadotropins (FSH and LH to stimulate maturation of multiple oocytes (e.g., Gonal F or Follistim or Menopur) at a range of 37.5-300 IU over 3 to 11 days, for example, at 150 IU/day over 3 days followed by 100 IU/day over 6 days. Alternatively, superovulation can be caused with oral medications such as clomiphene citrate (Clomid), or similar such as letrozole (Femara). Clomiphene citrate may be dosed at a range of 25-200 mg/day for 5-9 days. Letrozole may be dosed at a range of 2.5-12.5 mg/day for 5-9 days.
11. Trigger with human chorionic gonadotropin (hCG), at a range of 1,000-10,000 IU, for example, 5,000 IU, and/or Lupron (4 mg) when dominant follicle (DF) is at or above 10 mm in diameter. This can be confirmed by ultrasound to image the ovaries. The released oocytes are captured in the open end of the Fallopian tube and move towards the uterine cavity naturally after ovulation.
12. Approximately 36 hours after ovulation trigger, administer agent(s) to accelerate tubal transport. Agents used for accelerating tubal transport include Progesterone Receptor Blockers, Estradiol, or Prostaglandins individually or in any combination.
   a. Progesterone receptor blockers (i.e. mifepristone (Mifeprex)) can be administered at over a 1-3 day range, at a dosage range of 100-2,000 mg, by buccal administration.
   b. Estradiol (i.e. Estrace) can be administered at over 1-6 days, at a dosage range of 2-10 mg/day orally or by vaginal administration.
   c. Prostaglandins (misoprostol (Cytotec) and/or dinoprostone (Prepidil)) can be administered over 1-6 days, at a dosage range of 20-1600 mg, orally or by vaginal administration.
13. With the above compositions, tubal transport will be complete 10-84 hours after trigger and when the oocytes reach the uterus they will be mature and capable of fertilization. Progesterone receptor blockers, estradiol, and prostaglandins may be administered up to the day of the lavage procedure.
14. Perform uterine lavage approximately 10-108 hours after ovulation trigger. During the lavage Prostaglandins may be administered into the uterus to close the fallopian tube ostium.
15. If oocytes are immature, culture for in vitro maturation (IVM)
16. If a metaphase II (MII) oocyte is identified after uterine lavage, plan intracytoplasmic sperm injection (ICSI) with partner or donor sperm
17. Confirm fertilization by the identification of a zygote (single cell embryo). Embryos are further cultured to the blastocyst stage (5-7 days). Blastocysts are then biopsied for preimplantation genetic testing for aneuploidy and subsequent cryopreservation. Alternatively, non-invasive genetic and non-genetic tests can be performed using the culture media collected from IVF such as non-invasive preimplantation genetic testing for a aneuploidy, (NiPGT-A), Metabolomics, or Proteomics.
18. Embryo transfer of a previously cryopreserved euploid blastocyst for the establishment of an intrauterine pregnancy (at a later date).

Benefits of the Protocol

Oocytes recovered via uterine lavage can be fertilized for in vitro fertilization, or they can be cryopreserved for future use. Use of any of the above protocols protocol in combination with uterine lavage can provide a safer, less invasive alternative to oocyte aspiration. Uterine lavage addresses the current shortage of available reproductive services by allowing non-REs to collect oocytes with an office based procedure not requiring anesthesia. Thus, use of a novel stimulation protocol followed by uterine lavage has the potential to expand fertility care and lower cost.

In one embodiment of the present invention described above, a kit having instructions for use for performing uterine lavage in a female patient is disclosed which comprises a uterine lavage catheter configured for insertion into a woman's uterus to remove mature oocytes from the uterus; and at least a first container containing a sufficient dosage amount of an injectable gonadotropin (Gonal F or Follistim or Menopur) to induce superovulation and produce a plurality of mature oocytes in the women's uterus over a period of 3 to 11 days, wherein the kit does not include a GnRH antagonist. The injectable gonadotropin may be provided in a sufficient dosage amount to stimulate the ovaries and trigger the production of multiple oocytes over a shortened time period as compared to standard IVF protocols, e.g., over a period of 3 to 11 days. The kit may include instructions for use for administering the gonadotropins by self-injection, in which the dosage amount of injectable gonadotropin is appropriate for induction of superovulation, in vivo fertilization, and oocyte maturation. For example, the kit may include instructions for use for self-injecting the gonadotropin using 3 to 11 daily injections at ranges of 37.5 to 300 IU per day, e.g., at a total dosage amount of between 500 to 10000 IU's over the course of the superovulation protocol. The kit may further include one or more agents to accelerate tubal transport of the mature oocytes, wherein the one or more agents may comprise, e.g., a progesterone receptor blocker, estradiol, and/or a prostaglandin, for example, a progesterone receptor blocker at a dosage amount of between 100-2,000 mg, estradiol at a dosage amount of between 2-10 mg/day, and/or a prostaglandin at a dosage amount of between 20-1600 mg.

The kit may further include instructions for use for administering a single or multiple doses of urinary or recombinant hCG nasally, orally, subcutaneously, intramuscularly, transdermally, vaginally, rectally, or buccal route to induce ovulation. The kit may also include a second container or vial for storing and cryopreserving the recovered oocytes following the lavage procedure. Both of the one or more first and second containers may comprise an electronic or other coding label including the drug and any oocyte personal identification information.

The kit may further comprise instructions for use for performing at least one molecular diagnostic assay test on one or more of the recovered oocytes following recovery of oocytes from the uterus. The kit may comprise at least one instrument or agent for performing a biopsy from the one or more recovered oocytes. For example, the instrument may comprise a holding tool to stabilize one or more of the recovered oocytes during the biopsy procedure. The instrument may also comprise a control kit to ensure there is no contamination in the biopsied cells. The kit may further comprise a micropipette for performing the biopsy and removing the cells from the oocytes. The kit can include instructions for use for processing and handling (e.g., cryopreserving) the biopsied cells.

Other possible components of the kit include uterine lavage media, a consumable device which interfaces with an automated egg freezing machine such as the Gavi Automated Vitrification System (Geneabiomedx), cryogenic straws for storage of cryopreserved oocytes, a filter for facilitating the search for recovered oocytes following recovery from the uterus, an automated sperm processing kit for preparing sperm for IVF, a controller for controlling the pressure and vacuum applied to the uterine lavage catheter, catheter introducers, a pump and/or vacuum system and/or tubes, a cell culture vial for containing the oocytes following recovery and which maintains optimal CO2/O2 concentration for oocyte culture without the requirement of an incubator, or other cell-culture devices. The kit may be in packaged combination, such as in a pouch, bag or the like. The kit may further include instructions for the use of components of the kit in a uterine lavage procedure, such as instructions for use of the compounds to induce superovulation while stimulating the ovaries with injectable gonadotropins.

In another aspect of the invention, a composition suitable for administration to a human female patient during superovulation is disclosed to lead to the production of mature oocytes in the uterus of the patient. The composition may include human injectable gonadotropins (FSH and/or LH) in an amount sufficient to stimulate the ovaries at a dosage amount of about 500 to 10000 IU's over the course of treatment, e.g., in an amount sufficient to apply about 37.5-300 IU over 3 to 11 days. The injectable gonadotropins may include Gonal-F, Menopur, Novarel, Ovidrel, Pregnyl, Profasi, Chorex. Carriers may include aqueous solutions including saline, aqueous solutions including dextrose, water and buffered aqueous solutions, as well as oral formulations of the gonadotropins.

Methods are provided for administering an effective amount of the compositions (e.g., orally, nasally, intramuscularly, subcutaneously) to a patient to cause superovulation leading to the production of multiple mature oocytes in the uterus. For example, a method of inducing superovulation in a female patient to produce a plurality of mature oocytes in the uterus is disclosed which comprises administering injectable hCG to the patient to trigger superovulation, and gonadotropins that are administered daily for up to nine days or until the oocytes are at or between 8-18 mm in diameter as confirmed by ultrasound imaging of the ovaries. The method can include administering the gonadotropin over a period of between 3-11 days. For example, the method can include administering the gonadotropin over a period of three to six days. The gonadotropin may be administered at a dosage amount of between about 37.5 to 300 IU per day over a period of 3 to 11 days, e.g., the gonadotropin is administered at a dosage amount of about 150 IU per day over the first three days and 100 IU per day over the following 6-8 days.

What is claimed is:

1. A kit for uterine lavage, consisting of: a uterine lavage catheter configured for insertion into a woman's uterus to remove oocytes from the uterus; and one or more first containers with a sufficient dosage amount of an injectable gonadotropin over a period of 3 to 9 days to produce a plurality of mature oocytes in the women's uterus, wherein the kit does not include a GnRH antagonist.

2. The kit of claim 1, wherein the total dosage amount of the injectable gonadotropin to induce superovulation is between about 37.5 to 300 IU's per day.

3. The kit of claim 1, further comprising instructions for use for administering the injectable hCG trigger at a dosage of between about 1000 to 10000 IU's when dominant follicle is at or between 8-18 mm.

4. The kit of claim 3, further comprising instructions for use for administering the injectable gonadotropin at a dosage of about 150 IU's per day over the first three days and a dosage of about 100 IU's per day over the next six days.

5. The kit of claim 1, further including instructions for use for administering a single or multiple dose of urinary or recombinant hCG nasally, orally, subcutaneously, intramuscularly, transdermally, vaginally, rectally, or buccal route to trigger ovulation.

6. A kit for uterine lavage, comprising a uterine lavage catheter configured for insertion into a woman's uterus to remove mature oocytes from the uterus; one or more first containers with a sufficient dosage amount of an injectable gonadotropin to produce a plurality of mature oocyte, one or more second containers comprising a GnRH antagonist to prevent early ovulation, and one or more third containers comprising a progesterone receptor blocker, estradiol, and/or a prostaglandin to accelerate tubal transport of the mature oocytes.

7. A composition suitable for triggering and inducing superovulation in a female patient over a period of 3 to 11 days to produce a plurality of mature oocytes in the women's uterus, wherein the composition includes a dosage amount of an injectable gonadotropin of between about 500 to 10000 IU's.

8. A composition suitable for accelerating tubal transport of mature oocytes in a female patient over a period of 1 to 6 days, comprising a progesterone receptor blocker at a dosage amount of between 100-2,000 mg, estradiol at a dosage amount of between 2-10 mg/day, and/or a prostaglandin at a dosage amount of between 20-1600 mg.

9. A method of inducing superovulation in a female patient to produce a plurality of mature oocytes in the uterus of the patient, the method comprising administering an injectable gonadotropin to produce a plurality of oocytes wherein the gonadotropin is administered daily for up to eleven days or until the dominant follicle is at or between 8-18 mm in diameter as confirmed by ultrasound imaging of the ovaries.

10. The method of claim 9, including administering the gonadotropin over a period of between three to nine days.

11. The method of claim 10, including administering the gonadotropin over a period of three to six days.

12. The method of claim 9, wherein the gonadotropin is administered at a dosage amount of between about 37.5 to 300 IU per day over a period of three to 9 days.

13. The method of claim 12, wherein the gonadotropin is administered at a dosage amount of about 150 IU per day over the first three days and 100 IU per day over the following six days.

14. A method of retrieval of a plurality of mature oocytes from the uterus of a female patient, the method comprising administering an injectable gonadotropin to produce a plurality of oocytes wherein the gonadotropin is administered daily for up to eleven days or until the dominant follicle is at or between 8-18 mm in diameter as confirmed by ultrasound imaging of the ovaries, administering one or more agents to the female patient to accelerate tubal transport of the plurality of oocytes, and performing uterine lavage to remove the plurality of mature oocytes from the uterus of the patient.

15. The method of claim 14, wherein the one or more agents comprise a progesterone receptor blocker, estradiol, and/or a prostaglandin.

16. The method of claim 14, uterine lavage is performed approximately 10-108 hours after administering gonadotropin.
